(19) United States
(12) Patent Application Publication
(10) Pub. No.: US 2023/0324413 A1
(43) Pub. Date: Oct. 12, 2023

(54) RENIN-BASED ANALYSIS OF HEPCIDIN

(71) Applicant: TAMPERE UNIVERSITY FOUNDATION SR, Tampere University (FI)

(72) Inventors: Tarja KUNNAS, Tampere University (FI); Jarkko VALJAKKA, Tampere University (FI); Jaakko PIESANEN, Tampere University (FI); Seppo NIKKARI, Tampere University (FI)

(21) Appl. No.: 18/004,043

(22) PCT Filed: Jun. 21, 2021
(86) PCT No.: PCT/FI2021/050473

§ 371 Date: Dec. 30, 2022

(30) Foreign Application Priority Data
Jul. 2, 2020 (FI) 20205709

(51) Int. Cl. G01N 33/74

(52) U.S. Cl. CPC G01N 33/74; G01N 2333/96483; G01N 2410/02; G01N 2800/52

(57) ABSTRACT

Renin-based assays and kits for quantitative measurement of hepcidin, preferably biologically active hepcidin-25. Also provided are treatment modalities for the management of blood pressure and methods of determining or monitoring a subject's hepcidin status, status of a hepcidin-related disorder, and/or response to treatment by employing the hepcidin assay or kit provides novel treatment modalities for the management of blood pressure.

### TECHNICAL FIELD

The invention relates to the field of hepcidin analytics. More specifically, the invention provides novel means, assays and kits for quantitative measurement of hepcidin, preferably biologically active hepcidin-25, levels in a biological sample. Also provided are methods of determining or monitoring hepcidin status in a subject suffering from or suspected of suffering from a hepcidin-related disorder, or response to treatment by employing the hepcidin assay or kit. Moreover, the invention provides novel treatment modalities for the management of blood pressure.

### SEQUENCE LISTING

The Sequence Listing associated with this application has been submitted electronically via EFS-Web as an ASCII formatted text file. The name of the text file containing the sequence listing is HEPCIDIN_PCT_Sequence_Listing_PAP22305.txt. The text file was created on Jul. 2, 2020 and has a size of 2 KB. The ASCII formatted Sequence Listing contained in HEPCIDIN_PCT_Sequence_Listing_PAP22305.txt is part of the specification and is hereby incorporated by reference in its entirety.

### BACKGROUND

Hepcidin, a highly conserved peptide among different species, is an important circulating liver hormone linked to iron metabolism. By modulating hepcidin production, an organism regulates dietary iron absorption from the duodenum, controls the recycling of senescent erythrocyte iron by macrophages, and manages iron transport from hepatocytes into plasma for production of blood.

The mature bioactive form of hepcidin is a 25 amino acid peptide derived from a precursor of 84 amino acids (pre-pro-hepcidin) through two proteolytic cleavages. First, the 24 residue N-terminal signal peptide is cleaved to produce pro-hepcidin, which is then further processed to produce mature hepcidin, found in both blood and urine. Other existing hepcidin isoforms include smaller, N-terminally truncated isoforms consisting of 24, 23, 22 or 20 amino acids (hepcidin-24, -23, -22 and -20, respectively), and lacking 1, 2, 3 or 5 first N-terminal amino acids of hepcidin-25, respectively. Both hepcidin-25 and hepcidin-20 have been found in human serum, while human urine contains small amounts of hepcidin-22 in addition to the predominant hepcidin-25 and hepcidin-20. Other degradation products, such as hepcidin-24 and hepcidin-23, have been reported at undetectable or very low concentrations in human serum. The hepcidin isoforms other than the biologically active hepcidin-25 are of unknown significance, although they are generally present in diseases with elevated hepcidin-25 levels, including chronic kidney disease and sepsis.

Measurement of hepcidin levels in serum is thought to improve the understanding of disorders of iron metabolism. Indeed, comparisons of the measured hepcidin levels to normal levels is considered to be a useful tool in the differential diagnosis and clinical management of these diseases.

Unfortunately, however, the development of assays to measure hepcidin in biological samples has been challenging. Nonetheless, a number of hepcidin assays have been established. These assays can be divided into two major groups: mass spectrometry-based and classical immunoassays. Mass spectrometry (MS)-based methods, such as those disclosed in EP2057472 or Moe et al. (Clinical Chemistry, 2013, 59:1412-1414), can detect different hepcidin isoforms, but the complexity and the price of the equipment required limit the usefulness of the methods. On the other hand, immunoassay-based methods, such as competitive ELISA assays, generally monitor only total hepcidin levels, including pro-hepcidin, hepcidin-25 and its degradation products, making the assays non-selective for the biologically active hepcidin. However, some ELISA assays for hepcidin-25 appear to be now on the market. Nevertheless, results between different studies have varied considerably, and no common reference value for hepcidin is available. Therefore, comparing hepcidin-related studies is difficult.

Thus, there is an identified need for accurate hepcidin assays, especially for assays that are specific for the biologically active hepcidin-25.

### SUMMARY

In one aspect, the present invention provides use of renin in hepcidin analysis, including determining the presence or absence of hepcidin in a sample, as well as quantitating hepcidin in a sample.

In another aspect, the invention provides an assay for hepcidin analysis, comprising the following steps:
a) incubating angiotensinogen (AGT) and renin in the presence or absence of a sample whose hepcidin content is to be determined,
b) determining AGT cleavage by renin in the presence or absence of the sample,
c) detecting whether the sample inhibits the AGT cleavage, and
d) concluding that hepcidin is present in the test sample, if inhibition is detected.

In a further aspect, the invention provides use of a kit comprising AGT and renin for hepcidin analysis. Also provided is a kit for use in hepcidin analysis, comprising AGT, renin and at least one hepcidin control peptide comprising 5-25 consecutive amino acids from the N-terminus SEQ ID NO: 1.

In a still further aspect, the invention provides an isolated and/or synthetized hepcidin peptide consisting of 5-8 or 10-24 consecutive amino acids from the N-terminus SEQ ID NO: 1. Also provided is use of an isolated and/or synthetized hepcidin peptide comprising 5-25 consecutive amino acids from the N-terminus SEQ ID NO: 1 as a control peptide in renin-based hepcidin analysis.

In addition, the invention provides an in vitro method of determining a subject's hepcidin status, the method comprising the following steps:
a) quantitating hepcidin in a sample obtained from the subject by using the assay or the kit set forth above, and
b) determining said subject's hepcidin status based on the quantitated level hepcidin in the sample, wherein a hepcidin quantity lower than the normal hepcidin concentration is indicative of iron overload, preferably selected from anemias with iron overload, hereditary hemochromatosis, and ineffective erythropoiesis; whereas hepcidin quantity higher than the normal concentration of hepcidin is indicative of iron-restrictive anemias, preferably selected from iron-refractory iron deficiency anemias, resistance to erythropoietin and anemias associated with inflammation, chronic kidney disease, and some cancers.

In a further aspect, the invention provides an in vitro method of monitoring for a change in a subject's hepcidin status, the method comprising the following steps;
a) quantitating hepcidin in samples obtained from the subject at two or more time points, by using the assay or the kit set forth above,
b) a detecting a change in the quantitated hepcidin levels, if any, and
c) determining a change in the subject's hepcidin status from the detected change.

In a further aspect, the invention provides an in vitro a method of determining a subject's response to treatment, the method comprising the following steps:
a) quantitating hepcidin in samples obtained from the subject at two or more time points before, during or after the treatment, by using the assay or the kit set forth above,
b) a detecting a change in the quantitated hepcidin levels, if any, and
c) determining the subject's response to treatment or efficacy of the treatment from the detected change.

In a still further aspect, the invention provides hepcidin therapeutics for use in managing blood pressure or a disease or condition associated with abnormal blood pressure.

Further aspects, embodiments and details are set forth in following figures, detailed description, examples, and dependent claims.

### BRIEF DESCRIPTION OF THE DRAWINGS

The accompanying drawings illustrate several embodiments of the disclosed subject matter, and together with the description, serve to explain principles of the present assays, kits and methods.

FIG. 1 shows the amino acid sequences and molecular structures of hepcidin-25 and N-terminus of human angiotensinogen. The critical amino acid residues are shown in bold and highlighted by the frames.

FIG. 2 shows superimposed 3D structures of hepcidin-25 and N-terminus of human angiotensinogen peptide chains. Amino acid residues His, Phe, and Pro are shared by both structures.

FIG. 3 is a representation illustrating the accessibility of the renin activity site. Enlarged illustration of the activity site is shown on the left with a superimposed hepcidin-25 structure. Notably, hepcidin-25 can bind in the active site near the catalytic aspartates (framed) preventing angiotensinogen binding.

FIG. 4 shows the development of a fluorescence signal by renin-catalyzed substrate over time in the absence or presence of different concentrations of hepcidin (10 nM, 100 nM or 500 nM).

FIG. 5 illustrates the concentration dependent inhibitory effect of hepcidin on the activity of human renin. Mean inhibitory effects of hepcidin-25 on renin activities were 20.4%, 29.2% and 38.3% with concentrations of 10 nM, 100 nM and 500 nM, respectively. For hepcidin-9, the corresponding inhibitory effects were 41.25%, 41.15%, and 42.65%. For hepcidin-5, the corresponding inhibitory effects were 50.1%, 50.0% and 58.6%.

### DETAILED DESCRIPTION

The present invention can be understood more readily by reference to the following detailed description, examples, drawings, and claims, and their previous and following description. However, it is to be understood that this invention is not limited to any particular compositions, reagents, devices, protocols or methodology described, as such may vary. It is also to be understood that the terminology used herein is for the purpose of describing particular embodiments only and is not intended to limit the scope of the present invention, which will be limited only by the appended claims.

Any combination of the elements described herein in all possible variations thereof is encompassed by the invention unless otherwise indicated herein or otherwise clearly contradicted by context.

As used in the specification and in the appended claims, the singular expressions “a”, “an,” and “the” mean one or more. Thus, a singular noun, unless otherwise specified, carries also the meaning of the corresponding plural noun.

The present invention relates broadly to the use of renin in hepcidin analytics.

As used herein, the term “renin” (EC 3.4.23.15), also known as angiotensinogenase or angiotensin-forming enzyme, is a highly specific aspartic protease secreted by the kidneys and one of the key components of the renin-angiotensin cascade, a hormone system that regulates blood pressure and fluid balance. Renin mediates cleavage of angiotensin I peptide from the N terminus of angiotensinogen (AGT) between Leu10 and Val11 to release the N-terminal angiotensin I peptide. This peptide is subsequently processed by angiotensin-converting enzyme (ACE) to form angiotensin II, which is an important hormone that increases blood pressure. The effect is mediated through specific AT1 receptors, especially those present in the smooth muscles of vascular arteries. Angiotensin II also increases aldosterone secretion, as well as is involved in the activation of the sympathetic nervous system. Therefore, angiotensin II acts as an endocrine, autocrine/paracrine, and intracrine hormone.

In preferred embodiments, renin is human renin.

Notably, AGT is the only natural substrate of renin known to date.

It has now been surprisingly discovered that hepcidin is capable of binding to the active site of renin near the catalytic amino acids Asp38 and Asp226. Consequently, renin-mediated proteolytic cleavage of AGT can be inhibited by hepcidin as its presence prevents AGT from binding to renin. This unexpected realization opens new possibilities for hepcidin analytics.

As used herein, the term “hepcidin,” refers in particular to the mature bioactive form of hepcidin, i.e. a 25 amino acid peptide having the amino acid sequence set forth in SEQ ID NO: 1 and generally referred to as hepcidin-25 or hep-25 for short.

For molecules to undergo a binding reaction, they must collide and also take a relative orientation that enables them to bind together by noncovalent interactions. The amino acid sequence of hepcidin-25 (SEQ ID NO:1) has two regions which are involved in the binding to renin. The first region is a flexible N-terminal structure (amino acids 1-6 of SEQ ID NO: 1) which, when bound to the active site of renin, prevents renin's normal proteolytic activity. This critical part of the hepcidin structure is assisted into the active site by another compact fold (amino acids 7-25 of SEQ ID NO: 1) representing the second region important for binding to renin. Together these two regions provide suitable contacts for the interaction with renin.

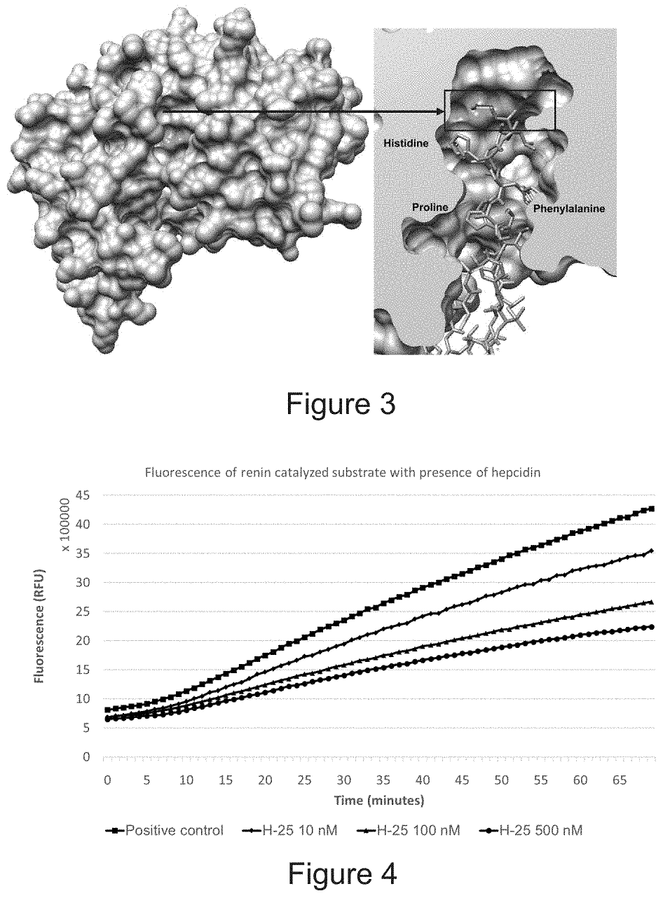

Renin inhibition by hepcidin-25 is competitive by nature because amino acids of hepcidin-25 crucial for the interaction with renin, i.e. His-Phe-Pro corresponding to amino acids 3-5 of SEQ ID NO: 1, can settle in the same places at the active site of renin as corresponding amino acids of AGT, the endogenous substrate of renin. Notably, respective orientation of these competing peptides is opposite as illustrated in FIG. 2. Binding of hepcidin-25 to the renin activity site is illustrated in FIG. 3.

Existing hepcidin isoforms other than the biologically active hepcidin-25 include smaller, N-terminally truncated isoforms consisting of 24, 23, 22 or 20 amino acids (hepcidin-24, -23, -22 and -20, respectively) and lacking 1, 2, 3 or 5 first N-terminal amino acids of hepcidin-25, respectively. Amino acid sequences of human hepcidin-24, -23, -22 and -20 are set forth in SEQ ID NOs: 2-5, respectively. Since the N-terminal amino acids crucial for the interaction with renin, i.e. amino acids corresponding to amino acids 3-5 of SEQ ID NO: 1, are missing from hepcidin-20 (SEQ ID NO: 5) and hepcidin-22 (SEQ ID NO: 4), it is unlikely that these isoforms would inhibit renin's proteolytic activity on AGT. In some embodiments, this may apply also to hepcidin-23 (SEQ ID NO: 3) lacking the two N-terminal amino acids preceding the crucial amino acid sequence His-Phe-Pro, and to hepcidin-24 (SEQ ID NO: 2) lacking the first N-terminal amino acid of hepcidin-25 corresponding to amino acid 1 of SEQ ID NO: 1.

In some embodiments, hepcidin is any natural hepcidin isoform, i.e. hepcidin-25 (SEQ ID NO: 1), hepcidin-24 (SEQ ID NO: 2), hepcidin-23 (SEQ ID NO: 3), hepcidin-22 (SEQ ID NO: 4) and/or hepcidin-20 (SEQ ID NO: 5). In some preferred embodiments, hepcidin is hepcidin-25 (SEQ ID NO: 1), hepcidin-24 (SEQ ID NO: 2) and/or hepcidin-23 (SEQ ID NO: 3). More preferably, hepcidin is biologically active hepcidin-25 (SEQ ID NO: 1).

Owing to the herein demonstrated capability of hepcidin-25 to prevent AGT from binding to the active site of renin, thereby preventing cleavage of AGT, hepcidin can be quantitated by employing renin, especially by means of assays or methods based on competitive inhibition. Basically, such assays or methods depend upon competition between two reactions. One reaction is a proteolytic reaction between AGT and renin, while the other reaction is a binding reaction between hepcidin and renin. The ratio of these reactions depends on the hepcidin quantity present. In other words, the degree of inhibition in the binding of AGT to renin, leading to inhibition in the cleavage of AGT by renin, is proportional to the amount of hepcidin in the reaction.

As used herein, the terms “assay” and “method” are interchangeable.

In accordance with the above, the present invention provides use of renin in detecting or quantitating hepcidin in a sample.

In some embodiments, hepcidin is hepcidin-25. Molecular modelling carried out in the context of the present invention indicates that the binding of the biologically inactive hepcidin isoforms to renin is significantly less likely than that of the biologically active hepcidin-25 or that of AGT, the natural substrate of renin. Hence, it is envisaged that the present means and assays for detecting or quantitating hepcidin are specific for hepcidin-25. In some embodiments, the present means and assays for detecting or quantitating hepcidin may detect or quantitate hepcidin-24 or hepcidin-23 in addition to the biologically active hepcidin-25. However, these inactive hepcidin isoforms are reported to exist in human samples at negligible concentrations.

As used herein, the term “quantitating hepcidin”, and any corresponding expressions, refer to quantifying or measuring the amount of hepcidin in a sample. The term “amount” is interchangeable with the terms “level” and “concentration”, and can refer to an absolute or relative quantity. Measuring hepcidin may also include simply determining the absence or presence of hepcidin in a sample.

As used herein, the term “sample” refers to any biological test sample suspected of containing hepcidin and that is to be analyzed for the presence of hepcidin or to be analyzed for the concentration of hepcidin. Preferred sample types are urine and blood, including whole blood samples, serum samples and plasma samples.

The present invention also provides assays for hepcidin analysis. These assays are to be performed in the presence of AGT, the natural substrate of renin. As explained above, hepcidin that is present in a sample whose hepcidin content is to be determined competes with AGT for binding to renin. The rate of inhibition in AGT's binding to renin, and subsequent cleavage of AGT by the proteolytic activity of renin, is directly proportional to the concentration of hepcidin in the sample.

At its simplest, the assay may be used only to detect the presence or absence of hepcidin in a test sample. In such embodiments, the assay may be expressed as an assay comprising the following steps:
a) incubating AGT, preferably human AGT, and renin, preferably human renin, in the presence or absence of the test sample,
b) determining AGT cleavage by renin in the presence or absence of the test sample,
c) detecting whether the test sample inhibits the AGT cleavage, and
d) concluding that hepcidin is present in the test sample, if said inhibition is detected.

Usually, however, the assay is used to quantitate hepcidin, i.e. to determine the concentration of hepcidin in a sample. In some embodiments, such an assay may be expressed, for example, as an assay for quantitating hepcidin in a test sample, wherein the assay comprises the following steps:
a) incubating AGT and renin in the presence or absence of the test sample,
b) determining capability of renin to cleave AGT in the presence or absence of the test sample,
c) calculating inhibition of AGT cleavage by the test sample, if any, and
d) quantitating hepcidin based on the calculated inhibition.

In some embodiments, determining an unknown concentration of hepcidin in a sample is based on previous measurements of standard solutions of known hepcidin concentrations. In other words, quantitating hepcidin may involve use of a calibration curve, i.e. a standard curve. Generating calibration curves is well known in the art and can in this case be carried out by plotting the rate of AGT cleavage versus the concentration of hepcidin that was applied in a given standard solution. Any unknown concentration of hepcidin may then be determined by comparing the detected rate of AGT cleavage to a corresponding rate on the calibration curve, achieved in the presence of known hepcidin concentrations.

In the assay, the AGT substrate is cleaved by renin to produce cleavage products, wherein a common property (e.g., ultraviolet absorbance or fluorescence) of the substrate and/or of one of the cleavage products is measured to determine the extent of cleavage. In other words, the AGT substrate, preferably a synthetic AGT substrate, contains a modification which, upon cleavage of the substrate by renin activity, creates a detectable signal.

Determining the rate of AGT cleavage by the proteolytic activity of renin, and inhibition of said cleavage by hepcidin or by a hepcidin-containing sample, can be carried out using any available or future protease assay technique suitable for this purpose.

Protease assays can be classified into homogeneous and heterogeneous assays. In homogeneous assays, all components of the reaction are present in an aqueous phase. In heterogeneous assays, one of the reaction components is immobilized on a solid surface, while the other components are in the aqueous phase. In some embodiments of heterogenous assays, the AGT substrate is immobilized, while renin and a test sample are provided in the aqueous phase. Non-limiting examples of heterogeneous assays include electrochemical assays, surface-enhanced Raman scattering (SERS) assays, and surface plasmon resonance (SPR) assays, as is well known in the art.

Homogeneous assays are usually preferred because they are easier to carry out and automate than heterogeneous assays. Moreover, in heterogeneous assay, any free reaction components must be physically separated from the immobilized substrate, e.g. by washings, while no such separation is necessary in homogeneous assays making homogeneous assays preferable.

Homogeneous assays of the invention include, but are not limited to, colorimetric assays, assays based on detection of ultraviolet signals and fluorescence resonance energy transfer (FRET) assays. Nanomaterials such as noble metal nanoparticles, quantum dots (QDs), and graphene oxide (GO) may also be employed in the assays as is well known in the art.

In an exemplary FRET-based assay of the invention, the AGT substrate is modified to contain a FRET donor and a FRET quencher in close proximity, such that the emission of the fluorescence donor is quenched by the acceptor (quencher). When the AGT substrate is cleaved by renin, the FRET quencher is separated from the FRET donor, which will emit a measurable fluorescent signal. The intensity of the signal proportional to the amount of AGT cleaved, which in turn is inversely proportional to the amount of hepcidin in the reaction (the concentration of AGT and renin being constant). Non-limiting examples of donor/quencher pairs known in the art and suitable for use in the present invention include fluorescein isothiocyanate (FITC)/tetramethylrhodamine isothiocyanate (TRITC), FITC/Texas Red™, FITC/N-hydroxysuccinimidyl-1-pyrenebutyrate (PYB)), FITC/eosin isothiocyanate (EITC), N-hydroxysuccinimidyl-1-pyrenesulfonate (PYS)/FITC, FITC/rhodamine X, and FITC/tetramethylrhodamine (TAMRA), fluorescein/rhodamine X, and Rhodamine X/Cy5. Non-fluorescent quencher molecules such as of 4-(dimethylaminoazo)benzene-4-carboxylic acid (DABCYL), DAMBI, 4-dimethylaminoazobenzene-4′-sulfonyl chloride (DABSYL) or methyl red may also be employed. For example, suitable donor/quencher pairs include fluorescein/DABCYL, 5-((2-Aminoethyl)amino)naphthalene-1-sulfonic acid (EDANS)/DABCYL, and EDANS/DABSYL.

In an exemplary Time Resolved Fluorescence Quenching Assay (TR-FQA) of the invention, the AGT substrate is modified to contain a TR donor and a quencher in close proximity, such that the emission of the fluorescent donor is quenched by the acceptor (quencher). When the AGT substrate is cleaved by renin, the quencher is separated from the donor, which will emit a measurable time-resolved fluorescent signal. Exemplary donors are luminescent lanthanide(III) chelates such as those disclosed in Anal. Biochem, 325 (2004), 317-325. Exemplary quenchers for use in TR-FQA assays are dabcyl, QSY-7, tetramethyl rhodamine, Alexa Fluor 546, and Cy-5.

In some embodiments, the AGT substrate is modified to undergo a detectable change in ultraviolet or visible absorbance when acted upon by renin.

The assay may be a fixed-time assay or a continuous assay. Continuous assays generally use a spectrophotometer to measure the appearance of a cleavage product, or disappearance of substrate in real-time. Spectrophotometric assays are simple, selective, and sensitive.

In some embodiments, the assay may be based on a binding reaction between renin and hepcidin in the absence of AGT. In such heterogeneous assays, renin is immobilized on a solid surface using means and methods readily available in the art. A biological test sample whose hepcidin content is to be determined is then contacted with the immobilized renin, and the rate of a binding reaction between renin and hepcidin is analysed by employing any appropriate assay technique available in the art including, but not limited to, electrochemical assay techniques, surface-enhanced Raman scattering (SERS) assay techniques, and surface plasmon resonance (SPR) assay techniques. In such embodiments, the rate of the binding reaction is proportional to the amount of hepcidin in the biological test sample.

The present invention also provides a kit and use thereof in quantitating hepcidin or determining its presence in a test sample. The kit comprises at least renin enzyme and AGT as the natural renin substrate. Preferably, human AGT and renin are used.

Both renin and AGT are available in the art, but they may also be produced using synthetic or recombinant techniques well known in the art. For example, an expression vector comprising a polynucleotide encoding for renin or AGT may be prepared by genetic engineering, and then transfected into a host cell for protein expression. Non-limiting examples of suitable host cells include prokaryotic hosts such as bacteria (e.g. E. coli, bacilli), yeast (e.g. Pichia pastoris, Saccharomyces cerevisiae), and fungi (e.g. filamentous fungi), as well as eukaryotic hosts such as insect cells (e.g. Sf9), and mammalian cells (e.g. CHO cells, HEK cells). Expression vectors may be transfected into host cells by a wide variety of techniques commonly used for the introduction of exogenous DNA into a prokaryotic or eukaryotic host cell including, but not limited to, electroporation, nucleofection, sonoporation, magnetofection, heat shock, calcium-phosphate precipitation, DEAE-dextran transfection and the like. A wide variety of expression vectors are readily available in the art, and those skilled in the art can easily select suitable ones depending on different variables, such as the host cell to be employed.

For homogeneous assays, AGT is modified to undergo a detectable change when acted upon by renin. In some embodiments, the change is a change in ultraviolet or visible light absorbance or a change in fluorescence emission. In some embodiments, the AGT substrate contains a FRET donor and a FRET quencher in close proximity as is disclosed in more detail above. Means and methods for synthetizing such a modified AGT substrate as well as coupling such a FRET pair to a recombinantly produced AGT are readily available in the art.

In some embodiments, the kit comprises synthetic hepcidin as a positive control peptide for testing that the kit or the assay to be carried out using the kit works as it should. In some embodiments, the control is hepcidin-25 or a fragment, preferably an N-terminal fragment, thereof. In some embodiments, the control peptide comprises or consists of 5-25, preferably 5-9 consecutive amino acids from the N-terminus of hepcidin-25 (SEQ ID NO: 1). To put it differently, the control peptide may comprise or consist of amino acids corresponding to those ranging from amino acids 1-5 of SEQ ID NO:1 to amino acids 1-25 of SEQ ID NO: 1. In some embodiments the control peptide comprises or consists of hepcidin-5 (SEQ ID NO: 6), hepcidin-6 (SEQ ID NO: 7), hepcidin-7 (SEQ ID NO: 8), hepcidin-8 (SEQ ID NO: 9) or hepcidin-9 (SEQ ID NO: 10). Control peptides may be created by any means, methods or techniques available in the art including, but not limited to, synthesis by an automated peptide synthesizer.

In some embodiments, the kit may also comprise one or more control samples with known hepcidin-25 concentrations to be used for generating a calibration curve. The number of control samples with different hepcidin-25 concentrations may vary as desired, but typically the kit comprises 3 to 5 control samples with different known hepcidin-25 concentrations. The concentration range may vary depending on different variables, such as type of the biological test sample whose hepcidin concentration is to be analysed with the kit (e.g. urine or blood) and the hepcidin-related disorder for whose management the kit is to be employed.

In some embodiments, the kit may also comprise one or more of hepcidin-20 (SEQ ID NO: 5), hepcidin-22 (SEQ ID NO: 4), hepcidin-23 (SEQ ID NO: 3) or hepcidin-24 (SEQ ID NO: 2), or one or more control samples comprising the same.

Preferably, the kit is provided in a form which enables storage of the kit in accordance with regulatory provisions generally applied to kits for clinical or research purposes. In some embodiments, one or more of said components are provided in a dry form, for example, as lyophilized compositions, and packaged to exclude moisture. Hence, the kit may also comprise one or more reconstitution buffers for re-dissolving the components and bringing them to specified concentrations. Preferably the reconstitution buffer is non-interfering (e.g., non-chelating, non-quenching, etc.), colourless and stable, as buffers usually are.

The kit may further comprise any appropriate additional components, depending on the detection principle to be employed or otherwise.

Furthermore, the present invention provides isolated and/or synthetized hepcidin peptides consisting of 5-8 or 10-24 consecutive amino acids from the N-terminus SEQ ID NO: 1, including for example hepcidin-5 (SEQ ID NO: 6), hepcidin-6 (SEQ ID NO: 7), hepcidin-7 (SEQ ID NO: 8) and hepcidin-8 (SEQ ID NO: 9) peptides. These peptides may be used, for example, as control peptides in hepcidin analysis, in addition to already known peptides hepcidin-9 (SEQ ID NO: 7) and hepcidin-25 (SEQ ID NO: 1). Preferably, the hepcidin analysis is renin-based hepcidin analysis of the present invention. Since renin-based hepcidin analysis has not been suggested earlier, the present invention also provides use of an isolated and/or synthetized hepcidin peptide comprising 5-25 consecutive amino acids from the N-terminus SEQ ID NO: 1, including for example hepcidin-5 (SEQ ID NO: 6), hepcidin-6 (SEQ ID NO: 7), hepcidin-7 (SEQ ID NO: 8), hepcidin-8 (SEQ ID NO: 9) and hepcidin-9 (SEQ ID NO: 10), as a control peptide in renin-based hepcidin analysis.

In some embodiments, isolated and/or synthetized hepcidin peptides provided herein consist of 6-8 or 10-24 consecutive amino acids from the N-terminus SEQ ID NO: 1, including for example hepcidin-6 (SEQ ID NO: 7), hepcidin-7 (SEQ ID NO: 8) and hepcidin-8 (SEQ ID NO: 9) peptides.

As used herein, the term “isolated” means that the given peptide is the predominant biological substance present, i.e. is substantially purified from other biological substances such as nucleic acids, lipids, cell remnants or other peptides, or any contaminating substances.

As used herein, the term “synthetized” means that the given peptide is produced by human action using technical means, e.g. by an automated peptide synthesizer.

Hepcidin plays a role in the pathogenesis of many disorders. Thus, the present invention provides a clinical tool for the management of hepcidin-related disorders, including but not limited to determining and/or monitoring hepcidin status or disease status in a subject suffering from or suspected of suffering from a hepcidin-related disorder, or response to treatment.

As used herein, the term “hepcidin status” refers to an absolute or relative amount of hepcidin in a sample obtained from a subject whose hepcidin status or disease status is to be determined. For example, a subject's hepcidin status can be higher than normal, lower than normal or normal. If compared to the amount of hepcidin in a sample obtained from the same subject's earlier, the subject's hepcidin status can be increased, decreased or unchanged.

As used herein, the term “disease status” refers to broadly any distinguishable manifestation of a disease, including non-disease. For example, the term includes, without limitation, information regarding the presence or absence of the disease, the presence or absence of a preclinical phase of the disease, the risk of the disease, the stage of the disease, and progression of the disease. Preferably, the disease is a hepcidin-related disorder.

As used herein, the term “hepcidin-related disorder” refers to any disease, disorder or pathological condition in which hepcidin is involved. Non-limiting examples of hepcidin-related disorders include disorders of iron metabolism and absorption, infectious diseases, inflammatory conditions, hypoxia-related disorders, cancers, and cardiovascular diseases. As is well known to those skilled in the art, these disease categories may overlap, and a given disease, disorder, or condition may belong to more than one category.

Being a key regulator of systemic iron homeostasis, hepcidin's unbalanced production contributes to the pathogenesis of many iron disorders. For example, pathologically increased hepcidin concentrations cause or contribute to iron-restrictive anemias, such as iron-refractory iron deficiency anemia, resistance to erythropoietin and anemias associated with inflammation, chronic kidney disease, and some cancers. On the other hand, hepcidin deficiency results in iron overload at least in anemia with iron overload, hereditary hemochromatosis, and ineffective erythropoiesis.

Transcription of hepcidin is upregulated during inflammation and infection. Abnormally high hepcidin levels result in a fall in serum iron due to iron trapping within macrophages and liver cells and decreased gut iron absorption. Inflammatory conditions include a vast array of disorders and conditions that are characterized by inflammation, whereas infectious diseases are caused by pathogenic microorganisms, such as bacteria, viruses, parasites, or fungi. Hepcidin-related inflammatory conditions include, but are not limited to, rheumatic diseases, inflammatory bowel disease and chronic infections, whereas non-limiting examples of hepcidin-related infectious diseases include sepsis.

Hypoxia refers to a condition in which oxygen is limited. During hypoxia, transcription of hepcidin is suppressed thereby enhancing intestinal iron uptake and release from internal stores. Hypoxia forms a key component of multiple diseases, including cardiovascular diseases, stroke, inflammatory diseases, degenerative disorders, and progression of solid tumors. Non-limiting examples of hypoxia-related disorders include lung diseases such as chronic obstructive pulmonary disease (COPD), emphysema, bronchitis, pneumonia, and pulmonary edema.

Notably, hypoxia and inflammation share an interdependent relationship. Just as hypoxia can elicit inflammation, inflamed tissues often become severely hypoxic. Thus, it is sometimes difficult to classify a certain disorder as an inflammatory condition or as a hypoxia-related disorder as these disease groups may overlap.

Local hepcidin levels are increased in many cancers, such as breast cancer, prostate cancer, lung cancer, multiple myeloma, non-Hodgkin lymphoma, colon cancer, and renal carcinoma, and appear to contribute to the metastatic invasion strategy of cancer cells and poor survival. Interestingly, manipulation of hepcidin expression to starve cancer cells for iron has been suggested as a potential new therapy in the anticancer arsenal.

Hepcidin plays a role also in cardiovascular diseases such as atherosclerosis, and blood pressure disorders including, but not limited to, hypertension (high blood pressure) and hypotension (low blood pressure), both of which have many causes and can range in severity from mild to dangerous. Without being limited to any theory, the present invention indicates that abnormal levels of hepcidin have an effect on blood pressure via renin inhibition.

As used herein, the term “subject” refers to an animal subject, preferably to a mammalian subject, more preferably to a human subject. The subject may or may not have been diagnosed with a hepcidin-related disorder.

For monitoring purposes, the method comprises analyzing and comparing at least two samples obtained from the same subject at various time points. The number and interval of the serial samples may vary as desired. The difference between the obtained assessment results serves as an indicator of a change in the subject's disease status and/or as an indicator of effectiveness or ineffectiveness of a treatment applied (i.e. as an indicator of response to a treatment), depending on the embodiment in question. In embodiments concerning monitoring for a response to treatment, the at least two samples to be analyzed may include samples taken before, during, or after treatment as appropriate, including at least one sample taken before and at least one sample taken during the course of the treatment; at least one sample taken before, at least one sample taken during the course of, and at least one sample taken after the treatment; at least one sample taken before and at least one sample taken after the treatment; at least two samples taken during the course of the treatment; and at least one sample taken during the course of and at least one sample after the treatment.

In some embodiments, taking of samples from the subject whose hepcidin status and/or response to treatment is to be determined or monitored is not part of the method, rendering the method as an in vitro method to be carried out with samples taken from the subject earlier.

The above described aspects of the invention may be expressed in different ways. For example, provided herein is a method, especially an in vitro method, of determining a subject's hepcidin status, the method comprising the following steps:
a) quantitating hepcidin in a sample obtained from the subject by using the present hepcidin assay or kit, and
b) determining said subject's hepcidin status based on the quantitated level hepcidin in the sample.

In some embodiments of the above method, hepcidin quantity (i.e. concentration) higher than the normal hepcidin concentration is indicative of a disorder associated with iron deficiency such as iron-refractory iron deficiency anemia (IRIDA), resistance to erythropoietin, anemia of inflammation, anemia in chronic kidney disease, or anemia associated with some cancers, whereas hepcidin quantity (i.e. concentration) lower than the normal concentration of hepcidin is indicative of a disorder associated with excessive iron load such as anemia with iron overload, hereditary hemochromatosis or ineffective erythropoiesis.

In some embodiments of the above method, hepcidin quantity higher than the normal hepcidin concentration contributes to or is indicative of a hepcidin-related disorder selected from the group consisting of inflammatory conditions such as rheumatic diseases, inflammatory bowel disease and chronic infections; infectious diseases such as sepsis; cancers, such as breast cancer, prostate cancer, lung cancer, multiple myeloma, non-Hodgkin lymphoma, colon cancer, and renal carcinoma; hypoxia-related disorders such as pulmonary edema, and cardiovascular diseases such as hypotension and atherosclerosis.

In some embodiments of the above method, hepcidin quantity lower than the normal hepcidin concentration contributes to or is indicative of a hepcidin-related disorder selected from the group consisting of hypoxia-related disorders including lung diseases such as chronic obstructive pulmonary disease (COPD), cardiovascular diseases such as hypertension.

As used herein, the term “normal hepcidin concentration” refers to the amount of hepcidin within reference values obtained from apparently healthy population.

Notably, the method does not involve therapeutic intervention but only provides help in clinical decision-making.

In some other implementations, the present method of determining a subject's hepcidin status may further include therapeutic intervention. For example, once a subject is identified as suffering from abnormally high hepcidin levels, the therapeutic intervention may include administration of inhibitors of hepcidin expression or hepcidin antagonists. On the other hand, once a subject is identified as suffering from hepcidin deficiency, he/she may be subjected to hepcidin replacement, e.g. by administration of hepcidin mimetics, inducers of hepcidin expression or inhibitors of ferroportin synthesis or activity. Alternatively or in addition, in the case of iron deficiency, an appropriate therapeutic intervention may include administration of oral or intravenous iron. On the other hand, in the case of iron overload, the subject may be subjected to phlebotomy, iron chelation therapy, or any other appropriate therapeutic intervention as the case may be.

Accordingly, in some embodiments, the present invention provides a method of determining hepcidin status and/or treating an hepcidin-related disorder in a subject in need thereof, the method comprising the following steps:
a) analyzing a sample obtained from the subject using a hepcidin assay or kit of the present invention,
b) determining whether the hepcidin concentration in the sample is abnormal or not, and
c) treating the subject based on the result obtained in step b).

Also provided is a method, especially an in vitro method, of monitoring for a change in a subject's hepcidin status or disease status, the method comprising the following steps;
a) quantitating hepcidin in samples obtained from the subject at two or more time points, by using the present hepcidin assay or kit,
b) a detecting a change in the quantitated hepcidin levels, if any, and
c) determining a change in the subject's hepcidin status or disease status from the detected change.

In some embodiments, no change in the hepcidin level may be indicative of no change in the subject's disease status. In diseases associated with increased or abnormally high hepcidin levels, further increased hepcidin levels may be indicative of progression or worsening of the disease, whereas decreased hepcidin levels towards normal levels may be indicative of amelioration or curing of the disease. Likewise, in diseases associated with decreased or abnormally low hepcidin levels, further decrease in the hepcidin levels may be indicative of progression or worsening of the disease, whereas increase in the hepcidin levels towards normal levels may be indicative of amelioration or curing of the disease.

In accordance with the above, the present invention also provides a method of determining a subject's response to treatment. This aspect of the invention may also be expressed as a method of determining efficacy of therapeutic intervention in a subject with hepcidin-related disease. In some embodiments, the method comprises the following steps;
a) quantitating hepcidin in samples obtained from the subject at two or more time points before, during or after the treatment, by using the present hepcidin assay or kit,
b) a detecting a change in the quantitated hepcidin levels, if any, and
c) determining the subject's response to treatment or efficacy of the treatment from the detected change.

In the method, treatment is determined as effective or the subject is determined as responsive to treatment, if the detected change (be it increase or decrease depending on the iron disease in question) is towards normal hepcidin levels. On the other hand, treatment is determined as ineffective or the subject is determined as nonresponsive, if no change towards normal hepcidin levels is detected.

In embodiments involving therapeutic intervention, the above method may be expressed as a method of determining efficacy of therapeutic intervention or response to treatment in a subject with hepcidin-related disorder, the method comprising the following steps:
a) subjecting the subject to therapeutic intervention,
b) quantitating hepcidin in samples obtained from the subject at two or more time points before, during or after the therapeutic intervention, by using the present hepcidin assay or kit,
c) a detecting a change in the quantitated hepcidin levels, if any, and
d) determining the subject's response to treatment or efficacy of the treatment from the detected change.

It is to be understood that while hepcidin measurements provide information that can be correlated with a subject's probable disease status or response to treatment, said disease status or response to treatment may not be finally determined on the basis of the present hepcidin measurements. In other words, in some embodiments, the method is not by itself determinative of the subject's disease status of response to treatment, but can indicate that further testing is needed or would be beneficial. Therefore, the present methods may be used in combination with one or more other diagnostic tests or markers for the final determination of the subject's disease status or response to therapeutic intervention.

The above-disclosed methods may also be expressed as use of the present assays and kits for corresponding purposes, namely for determining or monitoring a subject's hepcidin status, disease status, iron status and/or response to treatment.

Moreover, the present invention provides novel treatment modalities for the management of blood pressure and diseases or conditions related thereto. In view of the herein discovered link between hepcidin and renin-angiotensin cascade, hepcidin deficiency may, at least in some embodiments, contribute to hypertension while abnormally high hepcidin levels may contribute to hypotension. Thus, in some embodiments, hepcidin therapeutics such as hepcidin mimetics, inducers of hepcidin expression, or inhibitors of ferroportin synthesis or activity may be used for treating abnormal blood pressure, such as hypertension or disease or conditions involving hypertension. On the other hand, hepcidin therapeutics such as inhibitors of hepcidin expression or hepcidin antagonists may in some other embodiments be used for treating abnormal blood pressure, such as hypotension or diseases or conditions involving hypotension.

Accordingly, provided herein is a method of managing blood pressure in a subject in need thereof by administering an efficient amount of hepcidin therapeutics to said subject. This aspect of the invention may also be expressed as use of hepcidin therapeutics for managing blood pressure.

As used herein, the term “managing blood pressure” refers to the administration of hepcidin therapeutics to a subject in need thereof for purposes which may include treating or preventing abnormal blood pressure or a disease or condition associated with abnormal blood pressure.

As used herein, the term “abnormal blood pressure” refers either to abnormally high blood pressure, i.e. hypertension, or to abnormally low blood pressure, i.e. hypotension.

As used herein, the term “treatment” or “treating” refers to the administration of hepcidin therapeutics to a subject in need thereof for purposes which may include ameliorating, lessening, inhibiting, or curing abnormal blood pressure or a disease or condition associated with abnormal blood pressure; whereas the term “prevention” or “preventing” refers to any action resulting in suppression or delay of the onset of abnormal blood pressure or a disease or condition associated with abnormal blood pressure.

In some embodiments, the method of treating or preventing abnormal blood pressure, (i.e. either hypotension or hypertension as the case may be) or a disease or condition associated with abnormal blood pressure in a subject in need thereof comprises administering an efficient amount of hepcidin therapeutics to said subject.

As used herein, the term “efficient amount” refers to an amount by which harmful effects of abnormal blood pressure or a disease or condition associated with abnormal blood pressure are, at a minimum, ameliorated.

Amounts and regimens for administration of hepcidin therapeutics can be determined readily by those with ordinary skill in the clinical art of treating abnormal blood pressure and diseases or conditions associated thereto. Generally, dosing will vary depending on considerations such as: age, gender, and general health of the subject to be treated; kind of concurrent treatment, if any; frequency of treatment and nature of the effect desired; severity and type of disease or condition in question; causative agent of the disease, type of hepcidin therapeutics employed and other variables to be adjusted by the individual physician. A desired dose can be administered in one or more applications to obtain the desired results. For example, hepcidin therapeutics may be administered in a single daily dose, or the total daily dosage may be administered in divided doses of e.g. two, three or four times daily. Hepcidin therapeutics may be provided, for example, in unit dosage forms or in extended release formulations.

As used herein, the term “hepcidin therapeutics” refers to pharmaceutically acceptable agents which are either hepcidin agonists, i.e. substances that act like hepcidin or stimulate hepcidin expression or activity, or hepcidin antagonists, i.e. substances that inhibit expression or activity of hepcidin. Hepcidin agonists may be used for hepcidin replacement therapy.

Hepcidin agonists include, without limitation, hepcidin mimetics, inducers of hepcidin expression, and inhibitors of ferroportin activity. In accordance with the present invention, hepcidin agonists may be employed, for example, for the management, treatment, or prevention of abnormal blood pressure, including but not limited to management, treatment, or prevention of hypertension or for lowering high blood pressure.

Hepcidin mimetics include, but are not limited to, hepcidin derivatives such as synthetic endogenous human hepcidin (e.g. LJPC-401 by La Jolla Pharmaceutical Company) and PTG-300 by Protagonist Therapeutics Inc., as well as minihepcidins, i.e. short peptides based on the N-terminal amino acid segment of hepcidin that induce degradation of ferroportin. The first generation minihepcidins consist of 7 to 9 N-terminal amino acids with a free sulfhydryl group at C7 (e.g. Hep9) and derivatives thereof, such as retro-inverso analogues with or without conjugation to fatty acids (palmitoyl- groups) or chenodeoxycholic or ursodeoxycholic bile acids (cheno- and urso- groups, respectively). Further, non-limiting examples of minihepcidins include PR65, PR73, M004, M009, M012 and analogues thereof.

Potential inducers of hepcidin expression include, but are not limited to, recombinant Bone morphogenetic protein 6 (BMP6) and a wide range of natural or synthetic small molecules such as ipriflavone, vorinostat, diclofenac, icariin, resveratrol, querqetin, kaemferol, naringenin, epi-galoo-catechin-3-gallate, sorafenib, wortmannin, rapamycin, metformin, epimedin C, and adenine. In addition, hepcidin expression may be induced by agents that silence transmembrane protease serine-6 (Tmprss6), a suppressor of hepcidin production. Non-limiting examples of such agents include TMPRSS6-silencing oligonucleotides such as Tmprss6-antisense oligonucleotide by Ionis Pharmaceuticals Inc. and Tmprss6-siRNA by Alnylam Pharmaceuticals Inc.

In some embodiments, hepcidin replacement therapy may be achieved by inhibiting the synthesis or the iron-exporting activity of ferroportin. VIT-2763 by Vifor Pharma is a non-limiting example of small molecules that binds to ferroportin and inhibits iron efflux.

Hepcidin antagonists include, without limitation, direct hepcidin inhibitors, ferroportin-binding hepcidin inhibitors and inhibitors of hepcidin expression. In accordance with the present invention, hepcidin antagonists may be employed, for example, for the management, treatment or prevention of abnormal blood pressure, including but not limited to management, treatment or prevention of hypotension or for raising low blood pressure, e.g. upon blood pressure drop caused by sepsis.

Potential direct inhibitors of hepcidin include, without limitation, LY2787106, anticalins such as PRS-080, and spiegelmers such as NOX-H94; whereas potential ferroportin-binding hepcidin inhibitors include, without limitation, LY2928057, fursultiamine and quinoxaline.

Non-limiting examples of inhibitors of hepcidin expression include roxadustat; inhibitors of BMP6 and hemojuvelin (HVJ) such as LY3113593, heparin and derivatives thereof, erythroferrone, antibody-like fused protein sHJV.Fc, and monoclonal antibodies ABT-207 and H5F9-AM8; small molecule inhibitors of BMP/SMAD signaling such as dorsomorphin and its derivatives LDN-193189 and LDN-212854, myricetin, indazole-based inhibitors (e.g. DS28120313 and DS79182026), TP-0184, momelotinib, spironolactone, and imatinib; neutralizing antibodies against IL-6 receptor or IL-6 such as tocilizumab, MR16-1 and siltuximab; small molecule inhibitors of JAK/STAT3 signaling such as curcumin, AG490, PpYLKTK, acetylsalicylic acid, maresin 1, H₂P, metformin and guanosine 5′-diphosphate (GDP); sex hormones such as testosterone and 17β-estradiol; and vitamin D.

### EXAMPLES

### Example 1. Molecular Modelling

Crystal structure of the complex of human angiotensinogen (AGT) and renin was obtained from Protein Data Bank (PDB ID: 613F, Yan Y et al.). Crystal structure of hepcidin-25 was obtained from the same source (PDB ID: 1M4F, Hunter et al. (2002) J. Biol. Chem. 277: 37597-37603). The structure of the hepcidin-25 used in the modelling contained all four sulfur bridges and thus had its intrinsically functional intact molecular structure. In the modeling, programs proven to be reliable, such as Swiss-Model and CABS-dock, were used. Selected docking models were refined with molecular dynamics simulation, using NAMD on Sisu supercomputer at CSC—IT Center for Science Ltd.

Both hepcidin-25 and angiotensinogen peptides were docked together in the active site of renin to compare orientation and binding of hepcidin to that of the original substrate.

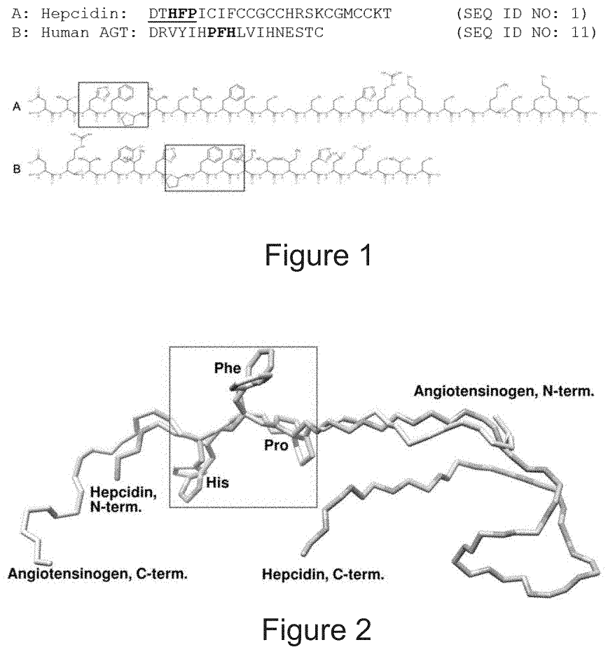

The modeling showed how hepcidin-25 binds to the active site of renin. As hepcidin binds, its stable structure containing sulfur bridges settles on the surface of renin such a way that the flexible N-terminal part is able to orient itself to the active site of renin. Molecular modelling showed that hepcidin-25 can bind in the active site of renin near the catalytic amino acids Asp38 and Asp226 preventing angiotensinogen binding. (FIGS. 1-3).

### Example 2. Interaction Analysis of Hepcidin Peptides

Human hepcidin-25/LEAP-1 (hep-25, with disulfide bonds between Cys7-Cys23, Cys10-Cys13, Cys9-Cys19, and Cys14-Cys22, purity assessed by HPLC≥98.0%) was purchased from Peptides International (Peptides International Inc, Louisville, KY). N-terminal part of hepcidin, DTHFPICIF (hep-9; SEQ ID NO: 6) and DTHFP (hep-5; SEQ ID NO: 7), were synthetized by Proteogenix (Schiltigheim, France). Renin inhibition screening kit was purchased from Biovision (Biovision, Inc. San Francisco, CA). ForteBio's 96 well black plates were used in the measurements.

Hep-25, hep-9, and hep-5 were used in final sample concentrations of 10 nM, 100 nM and 500 nM to measure inhibition of renin enzyme activity. The concentrations used represent normal levels of hepcidin in human circulation. Fluorescence measurement was performed using Perkin Elmer UV/VIS Envision multimode plate reader. Excitation and emission wavelengths were 328 and 552 nm, respectively. Fluorescence was recorded every 60 s for 60 min at 37° C. Rate of renin inhibition was calculated following the manufacture's instructions.

Results from fluorescence measurements in a kinetic mode using hep-25 as the renin inhibitor are shown in FIG. 4. Mean inhibitory effects of hep-25 on renin activity were concentration dependent being 20.4%, 29.2% and 38.3% with concentrations of 10 nM, 100 nM and 500 nM, respectively.

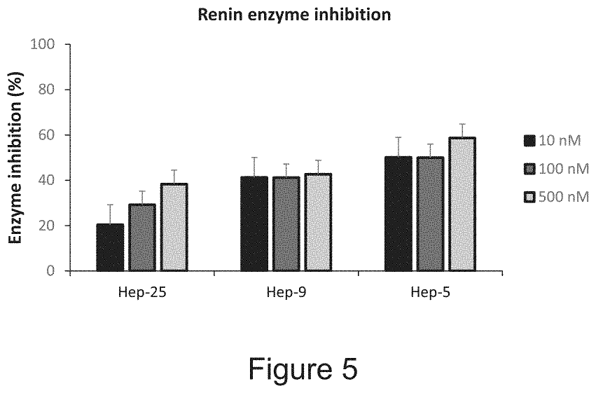

Moreover, N-terminal parts of hepcidin, namely hep-9 and hep-5, inhibited renin activity as expected (FIG. 5).

1. (canceled)

2. (canceled)

3. An assay for hepcidin analysis, comprising the following steps:
a) incubating angiotensinogen (AGT) and renin in the presence or absence of a sample whose hepcidin content is to be determined,
b) determining AGT cleavage by renin in the presence or absence of the sample,
c) detecting whether the sample inhibits the AGT cleavage, and
d) concluding that hepcidin is present in the sample, if inhibition is detected.

4. The assay according to claim 3, wherein step c) further comprises calculating inhibition rate of AGT cleavage by renin, if any, and step d) further comprises quantitating hepcidin based on the calculated inhibition rate.

5. The assay according to claim 3, wherein cleavage of AGT by renin results in a detectable signal.

6. The assay according to claim 5, further comprising correlating signal intensity to a calibration curve of known hepcidin concentrations to determine the presence or quantity of the hepcidin in the sample.

7. The assay according to claim 3, wherein hepcidin is hepcidin-25.

8. The assay according to claim 3, wherein the sample is a blood sample or a urine sample.

9. (canceled)

10. A kit for use in hepcidin analysis, comprising AGT, renin and at least one control peptide comprising 5-25 consecutive amino acids from the N-terminus SEQ ID NO: 1.

11. (canceled)

12. An isolated and/or synthetized hepcidin peptide consisting of 1-22 or 24 consecutive amino acids from the N-terminus SEQ ID NO: 1.

13. (canceled)

14. An in vitro method of determining a subject's hepcidin status, the method comprising the following steps:
a) quantitating hepcidin in a sample obtained from the subject by using the assay according to claim 3, and b) determining said subject's hepcidin status based on the quantitated level hepcidin in the sample, wherein hepcidin quantity lower than the normal hepcidin concentration is indicative of iron overload, preferably selected from anemias with iron overload, hereditary hemochromatosis and ineffective erythropoiesis, whereas hepcidin quantity higher than the normal concentration of hepcidin is indicative of iron-restrictive anemias, preferably selected from iron-refractory iron deficiency anemias, resistance to erythropoietin and anemias associated with inflammation, chronic kidney disease and some cancers.

15. An in vitro method of monitoring for a change in a subject's hepcidin status, the method comprising the following steps;
a) quantitating hepcidin in samples obtained from the subject at two or more time points, by using the assay according to claim 3,
b) a detecting a change in the quantitated hepcidin levels, if any, and
c) determining a change in the subject's hepcidin status from the detected change.

16. An in vitro a method of determining a subject's response to treatment, the method comprising the following steps;
a) quantitating hepcidin in samples obtained from the subject at two or more time points before, during or after the treatment, by using the assay according to claim 3,
b) a detecting a change in the quantitated hepcidin levels, if any, and
c) determining the subject's response to treatment or efficacy of the treatment from the detected change.

17. (canceled)

18. (canceled)


SEQUENCE LISTING (11 sequences)

SEQ ID NO: 1 - protein, 25 aa, Homo sapiens
DTHFPICIFCCGCCHRSKCGMCCKT

SEQ ID NO: 2 - protein, 24 aa, Homo sapiens
THFPICIFCCGCCHRSKCGMCCKT

SEQ ID NO: 3 - protein, 23 aa, Homo sapiens
HFPICIFCCGCCHRSKCGMCCKT

SEQ ID NO: 4 - protein, 22 aa, Homo sapiens
FPICIFCCGCCHRSKCGMCCKT

SEQ ID NO: 5 - protein, 20 aa, Homo sapiens
ICIFCCGCCHRSKCGMCCKT

SEQ ID NO: 6 - protein, 5 aa, Homo sapiens
DTHFP

SEQ ID NO: 7 - protein, 6 aa, Homo sapiens
DTHFPI

SEQ ID NO: 8 - protein, 7 aa, Homo sapiens
DTHFPIC

SEQ ID NO: 9 - protein, 8 aa, Homo sapiens
DTHFPICI

SEQ ID NO: 10 - protein, 9 aa, Homo sapiens
DTHFPICIF

SEQ ID NO: 11 - protein, 18 aa, Homo sapiens
DRVYIHPFHLVIHNESTC